(19) United States
(12) Patent Application Publication
(10) Pub. No.: US 2024/0082427 A1
(43) Pub. Date: Mar. 14, 2024

(54) METHOD OF DESIGNING HIGHLY REGULATED LENTIVIRAL VECTORS POSSESING STRICT ENDOGENOUS REGULATION

(71) Applicant: The Regents of the University of California, Oakland, CA (US)

(72) Inventors: Donald B. Kohn, Tarzana, CA (US); Ryan L. Wong, Los Angeles, CA (US); Roger Paul Hollis, Los Angeles, CA (US); Richard A. Morgan, Santa Monica, CA (US); Aaron Ross Cooper, Berkeley, CA (US)

(73) Assignee: The Regents of the University of California, Oakland, CA (US)

(21) Appl. No.: 18/263,325

(22) PCT Filed: Jan. 28, 2022
(86) PCT No.: PCT/US2022/014403

§ 371 Date: Jul. 27, 2023

Related U.S. Application Data
(60) Provisional application No. 63/143,663, filed on Jan. 29, 2021.

(51) Int. Cl. A61K 48/00; A61K 35/28; C12N 5/0789; C12N 15/86; G16B 35/20

(52) U.S. Cl. CPC A61K 48/005; A61K 35/28; C12N 5/0647; C12N 15/86; G16B 35/20; C12N 2740/16043; C12N 2830/008; C12N 2830/48

(57) ABSTRACT

In various embodiments method are provided for generating expression cassettes and gene therapy vectors comprising those cassettes that recapitulate the spatiotemporal pattern of expression of the endogenous gene. In certain embodiments the methods comprise (i) selecting a target gene; (ii) identifying putative regulatory elements associated with the target gene; (iii) determining if the regulatory element is a key regulatory element and (iv) providing a list of the key regulatory elements identified in step (iii).

### CROSS-REFERENCE TO RELATED APPLICATIONS

This application claims priority to and benefit of U.S. Ser. No. 63/143,663, filed on Jan. 29, 2021, which is incorporated herein by reference in its entirety for all purposes.

### STATEMENT OF GOVERNMENTAL SUPPORT

[Not Applicable]

### BACKGROUND

The field of virus-based gene therapy currently uses 3^{rd} generation lentiviral vectors, where expression of the transgene is typically driven by a selected internal promoter and/or regulatory element(s). When translating this design to the clinic, the goal has been to obtain therapeutic levels of expression from this internal promoter without having the genotoxicity associated with the previously used gamma-retro viral vectors.

This led to the development and use of many constitutively active promoter elements such as EFS1a (the promoter of the human elongation factor 1 alpha), the viral derived MNDU3 (see, e.g., Logan et al. (2004) Hum. Gene Ther. 15(10): 976-988), the promoter of the human Phosphoglycerate kinase (PGK) housekeeping gene, the promoter of the human Ubiquitin C (UBC) gene, as well as the use of ubiquitous chromatin opening element (UCOE) promoters (see, e.g., Zhang et al. (2010). Mol. Ther. 18(9): 1640-1649). Although, these approaches have displayed some success preclinically as well as clinically, a major limitation of using these constitutively active promoter elements is the lack of regulation and control of the gene being expressed. (especially as compared to the native gene).

### SUMMARY

Described herein are methods to design vectors to express transgenes that are controlled by their own endogenous regulatory elements and therefore possess strict lineage and temporal specific expression which is able to mimic the expression pattern of the native target gene of interest.

Various embodiments contemplated herein may include, but need not be limited to, one or more of the following:

Embodiment 1: A method of designing an expression cassette that recapitulates expression patterns for a target gene when introduced into a mammal, said method comprising:
(i) selecting a target gene;
(ii) identifying one or a plurality of putative regulatory elements associated with said target gene;
(iii) for each putative regulatory element identified in step (ii) determining if said regulatory element is a key regulatory element by:
   a) determining the genomic coordinates of said putative regulatory element to identify putative boundaries of said putative regulatory element;
   b) within said coordinates identifying a plurality of data tracks; and
   c) when the genomic coordinates of at least two of said data tracks overlap, identifying said regulatory element as a key regulatory element; and
(iv) providing a list of the key regulatory elements identified in step (iii).

Embodiment 2: The method of embodiment 1, wherein said selecting a target gene comprises selecting a gene associated with a genetic disorder.

Embodiment 3: The method according to any one of embodiments 1-2, wherein said target gene comprises a gene selected from the group consisting of β-globin gene, Factor IX (FIX), human fibroblast growth factor-4 (FGF-4), ND4, ABCD1, N-sulfoglucosamine sulfohydrolase (SGSH), REP1, CYBB, RAG1, ADA, WAS, AC6, Factor VIII, HGF (hepatocyte growth factor) HGF728 and/or HGF723, SMN, and CTRR.

Embodiment 4: The method according to any one of embodiments 1-3, wherein said identifying one or a plurality of putative regulatory elements associated with said target gene comprises querying one or more genomic databases that identify putative regulatory elements associated with genes identified in said one or more databases to identify said one or more putative regulatory elements.

Embodiment 5: The method of embodiment 4, wherein said identifying one or a plurality of putative regulatory elements associated with said target gene comprises querying one or more genomic databases selected from the group consisting of the ENCODE encyclopedia of DNA elements database, the Ensembl Regulatory Build database, the FANTOM5 atlas of active enhancers, the VISTA enhancer browser, the dbSUPER database of super enhancers, the eukaryotic promoter database (EPDnew), and the UCNEbase database, and/or database incorporating date from one or more of these databases.

Embodiment 6: The method of embodiment 5, wherein said identifying one or a plurality of putative regulatory elements associated with said target gene comprises querying a database incorporating data from a plurality of the databases.

Embodiment 7: The method of embodiment 6, comprises querying the GeneHancer database.

Embodiment 8: The method according to any one of embodiments 4-7, wherein said wherein said identifying one or a plurality of putative regulatory elements associated with said target gene comprises identifying a promoter.

Embodiment 9: The method according to any one of embodiments 4-8, wherein said wherein said identifying one or a plurality of putative regulatory elements associated with said target gene comprises identifying one or more enhancer(s).

Embodiment 10: The method according to any one of embodiments 1-9, wherein said determining the genomic coordinates of said regulatory element to identify putative boundaries of said regulatory element comprises retrieving said genomic coordinates from one or more genomic databases.

Embodiment 11: The method according to any one of embodiments 1-10, wherein said identifying a plurality of data tracks comprises inputting genomic coordinates of said putative enhancer into a genome browser and identifying overlapping data tracks in said genome browser.

Embodiment 12: The method of embodiment 11, wherein said genome browser comprises the UCSC genome browser.

Embodiment 13: The method according to any one of embodiments 1-12, wherein said identifying a plurality of data tracks comprises identifying a plurality of data tracks associated with gene expression and/or gene regulation.

Embodiment 14: The method according to any one of embodiments 1-13, wherein said plurality of data tracks comprise one or more data tracks selected from the group consisting of epigenetic histone modifications, chromatin looping interactions, DNAse I hypersensitivity site, transcription factor binding site, and conservation of sequence across a plurality of vertebrates.

Embodiment 15: The method according to any one of embodiments 1-13, wherein said plurality of data tracks comprise one or more data tracks selected from the group consisting of DNAse I hypersensitivity site, transcription factor binding site, and conservation of sequence across a plurality of vertebrates.

Embodiment 16: The method according to any one of embodiments 1-15, wherein said identifying a plurality of data tracks comprises identifying a DNAse I hypersensitivity site within said coordinates.

Embodiment 17: The method according to any one of embodiments 1-16, wherein said identifying a plurality of data tracks comprises identifying a transcription factor binding site within said coordinates.

Embodiment 18: The method according to any one of embodiments 1-17, wherein said identifying a plurality of data tracks comprises identifying a sequence conservation across a plurality of vertebrates.

Embodiment 19: The method according to any one of embodiments 1-18, wherein upon determining overlap of the data tracks DNAse I hypersensitivity site, transcription factor binding site, and conservation of sequence across a plurality of vertebrates, labeling regulatory element as a key regulatory element.

Embodiment 20: The method according to any one of embodiments 1-19, wherein said conservation of sequence across a plurality of vertebrates comprises 100 vertebrates sequence conservation.

Embodiment 21: The method according to any one of embodiments 1-20, where said providing a list comprises providing the genomic coordinates of each of the key regulatory elements comprising said list.

Embodiment 22: The method of embodiment 21, wherein said providing a list comprises providing output in a form selected from the group consisting of hard copy, data in a non-transitory computer readable memory, presentation of data on a display, provision of a datafile readable by a computer, provision of a data file readable by a DNA synthesizer. And output readable by a DNA vector design program.

Embodiment 23: The method of embodiment 22, wherein said providing a list comprises providing output readable by a DNA vector design program.

Embodiment 24: The method of embodiment 22, wherein said providing a list comprises providing output readable by a DNA vector design program selected from the group consisting of GENESCRIPT®, VECTOR NTI®, GENSMART® DESIGN, BENCHLILNG, and SNAPGENE®.

Embodiment 25: The method according to any one of embodiments 1-24, wherein said method comprises assembling an expression cassette where said expression cassette comprises:
a gene or cDNA to be expressed by said cassette;
a promoter comprising an endogenous promoter for said gene, or a reduced promoter comprising key regulatory elements comprising said promoter; and
one or more key regulatory elements that are not elements of said promoter selected from said list of key regulatory elements.

Embodiment 26: The method of embodiment 25, wherein said expression cassette comprise an endogenous promoter for said gene.

Embodiment 27: The method of embodiment 25, wherein said expression cassette comprises a reduced promoter comprising key regulatory elements comprising said promoter.

Embodiment 28: The method according to any one of embodiments 25-27, wherein said expression cassette comprises a key regulatory element comprising an enhancer where said enhancer is disposed upstream from said promoter.

Embodiment 29: The method according to any one of embodiments 25-28, wherein said gene or cDNA comprises a reporter gene.

Embodiment 30: The method of embodiment 29, wherein said reporter comprises a reporter gene selected from the group consisting of mcitrine, mStrawberry, green fluorescent protein (GFP), yellow fluorescent protein (YFP), and red fluorescent protein (RFP).

Embodiment 31: The method according to any one of embodiments 25-28, wherein said gene or cDNA comprises said target gene or a cDNA of said target gene, or a modified form of said target gene or target gene cDNA.

Embodiment 32: The method of embodiment 31, wherein said gene or cDNA comprises a gene selected from the group consisting of β-globin gene, anti-sickling β-globin (βAS3-FB), Factor IX (FIX), human fibroblast growth factor-4 (FGF-4), ND4, ABCD1, N-sulfoglucosamine sulfohydrolase (SGSH), REP1, CYBB, RAG1, ADA, WAS, AC6, Factor VIII, HGF (hepatocyte growth factor) HGF728 and/or HGF723, SMN, CTRR, BTK, ILR2-G, CD40L, Il7Rg, CD3 delta, CD3 epsilon, CD3 zeta, ZAP70, and FOXP3.

Embodiment 33: The method according to any one of embodiments 25-32, wherein said method comprises providing said expression cassette in a gene therapy vector.

Embodiment 34: The method of embodiment 33, wherein method comprises providing said expression cassette in a gene therapy vector selected from the group consisting of a lentiviral vector (LV), an adenovirus vector (AV), and an adeno-associated viral vector (AAV).

Embodiment 35: The method of embodiment 33, wherein said gene therapy vector is a lentiviral vector.

Embodiment 36: The method of embodiment 35, wherein said vector is an HIV-1 lentiviral vector.

Embodiment 37: The method according to any one of embodiments 33-36, wherein said gene therapy vector includes a known nucleotide sequence in the 3′ untranslated region of said vector where a unique known DNA barcode sequence is provided for each putative enhance in said expression cassette.

Embodiment 38: The method according to any one of embodiments 33-37, wherein said vector is introduced into a mammalian cell and the expression level of the gene or cDNA encoded in the vector is determined.

Embodiment 39: The method of embodiment 38, wherein said vector is transduced into a primary cell line.

Embodiment 40: The method of embodiment 38, wherein said vector is transduced into a cell line.

Embodiment 41: The method of according to any one of embodiments 38-40, wherein said method comprises quantifying expression of said gene or cDNA in said primary cell or cell line where elevated level of expression of said gene indicates that said putative enhancer(s) are valid/effective enhancer(s).

Embodiment 42: The method of embodiment 41, wherein said method comprises quantifying expression of a reporter gene.

Embodiment 43: The method of embodiment 41, wherein said method comprises quantifying expression of a reporter gene using flow cytometry.

Embodiment 44: The method of embodiment 38, wherein said vector is transduced into a cell that is transplanted back into a test mammal.

Embodiment 45: The method of embodiment 44, wherein said vector is transduced into a hematopoietic stem cell (HSC).

Embodiment 46: The method of embodiment 45, wherein said vector is transduced into a CD34+ hematopoietic stem cell.

Embodiment 47: The method according to any one of embodiments 44-46, wherein said vector is transduced into a mammal selected from the group consisting of a mouse, a rat, a rabbit, a porcine, a canine, a camelid, and a non-human primate.

Embodiment 48: The method of embodiment 47, wherein said vector is transduced into a mouse.

Embodiment 49: The method of embodiment 48, wherein said vector is transduced into an NSG or a BLT mouse.

Embodiment 50: The method according to any one of embodiments 44-49, wherein cells or tissues or organs are harvested from said animal at one or more time points after transduction of said vector into said mammal quantification of vector nucleic acid in said cells, tissues, or organs to quantify the activity of the putative enhancer(s) in each cell population where an elevated expression level of said vector compared to the genomic DNA level indicates that said putative enhancer(s) are valid/effective enhancer(s).

Embodiment 51: The method of embodiment 50, wherein said method comprises extracting genomic DNA and RNA, converting the RNA to cDNA, and using DNA amplification to amplify barcodes from the gDNA and RNA and quantifying the abundance of bar codes in the RNA relative to the gDNA.

Embodiment 52: The method of embodiment 50, wherein said method comprises direct single cell RNA sequencing of cells from said mammal to identify cellular identity and to quantify the abundance of barcodes in the transcriptome of said cell.

Embodiment 53: The method according to any one of embodiments 41 and 50, wherein said method comprises viewing data tracks for the enhancers identified as valid/effective enhancers and defining minimal boundaries for each of the enhancer(s) to produce slimmed enhancers.

Embodiment 54: The method of embodiment 53, wherein said minimal boundaries are defined by a DNaseI hypersensitivity data tracks.

Embodiment 55: The method of embodiment 53, wherein said method comprises placing one or more of said slimmed enhancers upstream of said promoter or reduced promoter to generate a lead candidate vector.

Embodiment 56: A vector designed according to the method according to any one of embodiments 1-55.

Embodiment 57: The vector of embodiment 56, wherein said vector is a lentiviral vector.

### Definitions

The term “regulatory element” or “gene regulatory element” refers to genomic sequences that control spatiotemporal patterns of gene expression. Alterations to gene regulatory elements are often associated with inter-individual phenotypic variation and human disease. Illustrative regulatory elements include but are not limited to enhancers and promoters.

A “promoter” refers to a sequence of DNA needed to turn a gene on or off. The process of transcription is initiated at the promoter. Usually found near the beginning of a gene, the promoter typically has a binding site for the enzyme used to make a messenger RNA (mRNA) molecule.

Enhancers” refers to Enhancers are cis-regulatory DNA sequences that are widely dispersed throughout genomes. Enhancers are distant-acting transcription factor (TF)-binding elements able to modulate target gene expression in a precise spatiotemporal specific manner (see, e.g., Marsman & Horsfield (2012) Biochim. Biophys. Acta, 1819: 1217-1227; Levo & Segal (2014) Nat. Rev. Genet., 15: 453-468). There is considerable evidence that enhancer-based transcription regulation is involved in determining cell fate and tissue development (see, e.g., Bonn et al. (2012) Nat. Genet., 44: 148-156; Taminato et al. (2016) Genomics, 108: 102-107). The accepted model for enhancer-mediated activation of gene expression is that enhancers come into proximity with promoters by chromatin looping, thus recruiting the transcriptional machinery (see, e.g., Marsman & Horsfield (2012) Biochim. Biophys. Acta, 1819: 1217-1227; Blackwood & Kadonaga (1998) Science, 281: 60-63; Bulger & Groudine (1999) Genes Dev., 13: 2465-2477; de Laat et al. (2008) Curr. Top. Dev. Biol., 82: 117-139). This mode of action is supported by chromosome conformation capture and related methods that detect direct interactions among remote chromatin regions (see, e.g., Dixon et al. (2016) Mol. Cell, 62: 668-680). It is estimated that there are hundreds of thousands of enhancers in the human genome (see, e.g., Pennacchio et al. (2013) Nat. Rev. Genet., 14: 288-295), a count much larger than that of genes. Each enhancer binds several TFs, consistent with a combinatorial regulatory code (see, e.g., Duque & Sinha (2015) Genome Biol. Evol., 7: 1415-1431), likely involving many-to-many relationships among enhancers and genes (see e.g., Yao et al. (2015) Crit. Rev. Biochem. Mol. Biol., 50: 550-573).

A “genetic disorder” refers to a disease caused in whole or in part by a change in the DNA sequence or expression pattern away from the normal sequence or expression pattern. Genetic disorders can be caused by a mutation in one gene (monogenic disorder), by mutations in multiple genes (multifactorial inheritance disorder), by a combination of gene mutations and environmental factors, or by damage to chromosomes (changes in the number or structure of entire chromosomes, the structures that carry genes).

A gene associated with a genetic disorder refers to a gene where altered expression of said gene and/or alterations in the amount or sequence of expression product of said gene as compared to a wildtype expression product for said gene are causal of, or a causal component of, a genetic disorder.

A Data track is a genomic region possessing the features delineated by the data track. Thus, for example, a DNase I hypersensitivity data track is a genomic region encoding a predictive or experimentally determined DNase hypersensitivity region. A transcription factor track is a genomic region encoding a predictive or experimentally determined transcription factor binding site. A sequence conservative data track, is a genomic region of DNA containing predictive or experimentally determined conserved sequence across different species. These examples are not limiting.

A “DNA barcode sequence” or “DNA barcode” refers to a DNA sequence that can be uniquely recognized. Similarly, an “RNA barcode sequence” or “RNA barcode” refers to an RNA sequence that can be uniquely recognized. The barcode can be used to uniquely identify the nucleic acid construct within which that barcode sequence is disposed. As used herein DNA barcodes typically range in length from about 10 to about 20 base pairs, however they can theoretically be essentially any size.

An expression cassette refers to a component of vector DNA comprising a gene to be expressed by a transfected or transduced cell and a regulatory sequence that controls expression of that gene.

A “key regulatory element” refers to a regulatory element or component thereof necessary to recapitulation of the expression pattern of the endogenous gene.

### BRIEF DESCRIPTION OF THE DRAWINGS

FIG. 1 illustrates the contents of GeneCard from GeneHancer database.

FIG. 2 illustrates screenshot of the putative genomic regions regulating the CYBB gene from the GeneHancer database. There was a total of 25 regulatory regions, but this figure just shows the top 9 on the screen.

FIG. 3 illustrates data shown by GeneHancer for a putative regulatory element for the CYBB gene.

FIG. 4 illustrates the visual interface of the UCSC Genome Browser for the genomic coordinates of one putative regulatory element. Also shown are data tracks used to identify key regulatory elements.

FIG. 5 illustrates the identification of two key regulatory elements within a larger genomic regions.

FIG. 6 illustrates various lentiviral (LV) constructs used to validate putative enhancer elements.

FIG. 7 illustrates determination of the endogenous expression pattern for the CYBB gene.

FIG. 8 illustrates screening of the lentiviral constructs in a mature neutrophil population. 15 single element vectors are shown. MSP is the currently clinical vector, and Int3-pro-mCit-WPRE is the parental backbone to which each of putative elements were attached.

FIG. 9 illustrates screening of the lentiviral constructs in monocytes. 15 single element vectors are shown. MSP is the currently clinical vector, and Int3-pro-mCit-WPRE is the parental backbone to which each of putative elements were attached.

FIG. 10 illustrates screening of the lentiviral constructs in B cells. 15 single element vectors are shown. MSP is the currently clinical vector, and Int3-pro-mCit-WPRE is the parental backbone to which each of putative elements were attached.

FIG. 11 illustrates screening of the lentiviral constructs in T-cells. 15 single element vectors are shown. MSP is the currently clinical vector, and Int3-pro-mCit-WPRE is the parental backbone to which each of putative elements were attached.

FIG. 12 illustrates the structure of a lentiviral vector that recapitulates endogenous expression of the CYBB gene.

### DETAILED DESCRIPTION

In various embodiments methods are provided to design vectors to express transgenes that are controlled by their own endogenous regulatory elements and therefore possess strict lineage and temporal specific expression which is able to mimic the expression pattern of the native target gene of interest.

With the use of 3rd generation lentiviral vectors, expression of the transgene is now driven by an internal promoter and desired regulatory elements. Many pre-clinical and clinical lentiviral vectors to date use constitutively active promoter elements (e.g., EFS-1a, MNDU3, PGK, UBC, UCOE) in an attempt to drive therapeutic levels of expression of the transgene but these vectors lack any sort of lineage or temporal specific expression. However, tightly regulated expression of a transgene is important in order to prevent toxicity associated with aberrant expression of certain genes (eg. CD40L, BTK, RAG-1/2).

The methods described herein facilitate the design of vectors to express transgenes that are controlled by their own endogenous regulatory elements and therefore possess strict lineage and temporal specific expression which is able to mimic the expression pattern of the native target gene of interest.

In one illustrative, but non-limiting embodiment, putative enhancer element(s) regulating a target gene of interest are data mined from a publicly available database (e.g., the GeneHancer database). The genomic coordinates of each putative enhancer input into a genome browser (e.g., the UCSC Genome Browser) visual interface and using imported data tracks such as cell specific DNaseI Hypersensitivity, Transcription factor binding by ChIP-seq, Epigenomic modifications and sequence conservation, the general boundaries of each putative enhancer element are identified as well as the native promoter of interest.

In various embodiments, a series of lentiviral vectors can be then be created, with each vector having a single putative enhancer element (or a specific combination of putative enhancer elements (e.g., at least 2 enhancer elements, at least 3 enhancer elements, at least 4 enhancer elements, or at least 5 putative enhancer elements) placed upstream of the promoter of interest (e.g., the native promoter or a reduced native promoter) to drive expression of a gene or cDNA (e.g., a reporter gene).

In certain embodiments where multiple putative enhancers are identified, an enhancer library can be produced by generating vectors where a known nucleotide sequence is added to the 3′ untranslated region or the vector, thereby associating each putative enhancer element to a specific barcode. This library (e.g., lentiviral library) can then be screened, for example, in cell lines, in primary cells, or by transducing human CD34+ hematopoietic stem cells (HSCs) and transplanting them into a mammalian test animal (e.g., NSG (NOD.Cg-Prkdcscid Il2rgtm1Wjl/SzJ) or BLT mice. In the case of transplanting the transduced HSCs into a subject (e.g., NSG/BLT mice), target organs or tissue(s) of interest (e.g., bone marrow, spleen, thymus, liver, peripheral blood, etc.) can be harvested post-transplant (e.g., at 16-20 weeks) and different stage specific lineages can be FACS sorted. Genomic DNA (gDNA) and RNA can then be extracted from each population and the RNA can be cDNA converted which facilitates the use of PCR (or other DNA amplification systems) to amplify out the barcodes from the gDNA and the RNA. By quantifying the abundance of barcodes in the RNA relative to the gDNA, the activity of each putative enhancer element in each cell population can be quantified.

In another illustrative, but non-limiting embodiments, an alternative method is to quantify the barcodes associated with each enhancer by skipping the FACs sorting and PCR steps and directly performing single cell RNA sequencing of the cells from each target organ. By RNA sequencing, it is possible to identify the cellular identity of each cell as well as quantify the abundance of barcodes in the transcriptome.

A lower throughput method of screening is to use flow cytometry to evaluate the expression of reporter gene in each construct across cell lines and/or primary cells. (this does not require barcodes).

After putative enhancer elements have been screened, the sequences of the validated enhancers of interest are then inputted back into a genome browser (e.g., the UCSC genome browser) and minimal boundaries are then defined (usually by DNaseI Hypersensitivity footprint). These “slimmed” enhancer elements that have been reduced in size are then compared to their original larger fragments to evaluate if enhancer activity has been retained. The enhancer element(s) (and in certain embodiments the promoter) are reduced in size with the goal of increasing titer while retaining expression. The validated and slimmed enhancer elements can then be combined together upstream of the minimal promoter of interest to generate a lead candidate vector for the disease of interest.

More specifically, in certain illustrative, but non-limiting embodiments, methods of designing (and optionally constructing) an expression cassette that recapitulates expression patterns for a target gene when introduced into a mammal, are provided where the methods comprise:
(i) selecting a target gene;
(ii) identifying one or a plurality of putative regulatory elements associated with said target gene;
(iii) for each putative regulatory element identified in step (ii) determining if said regulatory element is a key regulatory element by:
   a) determining the genomic coordinates of said regulatory element to identify putative boundaries of said regulatory element; and
   b) within said coordinates identifying a plurality of data tracks (e.g., expression-related data tracks); and
   c) when the genomic coordinates of at least two of the data tracks overlap, identifying said regulatory element as a key regulatory element; and
(iv) providing a list of the key regulatory elements identified in step (iii).

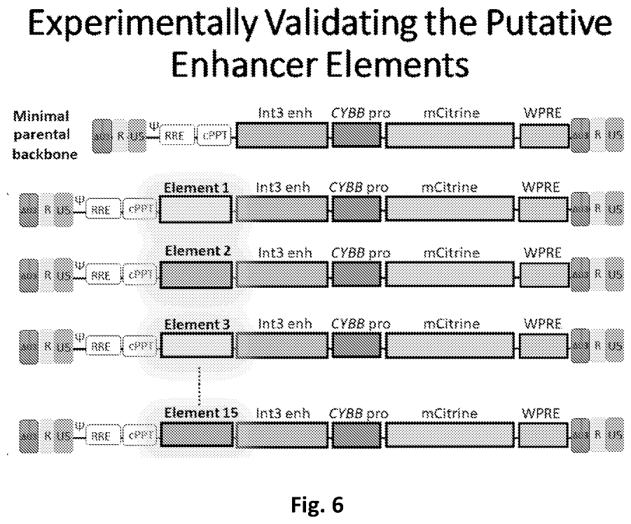

As described below, the key regulatory elements can be used in the construction of an expression cassette and a vector comprising the expression cassette for validation in an appropriate biological model (see, e.g., FIG. 6).

These various steps are further described below.

### (i) Selecting a Target Gene.

The methods described herein can readily be used to identify the regulatory elements necessary to construct an expression cassette that recapitulates the endogenous expression pattern of essentially any gene of interest (“target gene”). In certain embodiments the target gene comprise a gene associated with a genetic disorder. In certain embodiments the target gene is one for which dysregulation of expression of the gene and/or expression of an altered gene product is causal of a pathology or is a contributory factor in a pathology. In certain embodiments the target gene is one where restoration of normal expression of a “normal” or “corrective” gene product ameliorates one or more symptoms of a pathology or reduces or eliminates that pathology.

Illustrative, but non-limiting examples of genetic diseases and the associated genes are shown in Table 1.

TABLE 1
Illustrative, but non-limiting examples of genetic disease.

| Pathology | Gene |
|---|---|
| B-thalassemia | β-globin gene |
| Sickle cell disease | Normal β-globin or |
| | anti-sickling β-globin |
| | (βAS3-FB) |
| Hemophilia | Factor IX (FIX) |
| Refractory angina, Cardiac syndrome X, | human fibroblast growth |
| Congestive heart failure, Moyamoya disease | factor-4 (FGF-4) |
| Leber Hereditary Optic Neuropathy (LHON) | ND4 |
| Cerebral adrenoleukodystrophy (CALD) | ABCD1 |
| Mucopolysaccharidosis IIIA (MPS IIIA), | N-sulfoglucosamine |
| also known as Sanfilippo type A syndrome | sulfohydrolase (SGSH) |
| Choroideremia | REP1 |
| X-linked chronic granulomatous disease | CYBB |
| (X-CGD) | |
| Recombination-activating gene 1 (RAG1) | RAG1 |
| severe combined immunodeficiency (SCID) | |
| Adenosine deaminase severe combined | ADA |
| immune deficiency (ADA-SCID) | |
| Wiskott Aldrich syndrome (WAS) | WAS |
| Heart failure and reduced ejection fraction | AC6 |
| (HFrEF) | |
| Hemophilia A | Factor VIII |
| Painful diabetic peripheral neuropathy; | HGF (hepatocyte growth |
| Chronic nonhealing ischemic foot ulcer | factor), HGF728, HGF723 |
| in diabetes; Critical limb ischemia]; | |
| Amyotrophic lateral sclerosis; Acute | |
| myocardial infarction | |
| Spinal muscular atrophy (SMA) | SMN |

In certain embodiments the target gene comprises a gene selected from the group consisting of β-globin gene, Factor IX (FIX), human fibroblast growth factor-4 (FGF-4), ND4, ABCD1, N-sulfoglucosamine sulfohydrolase (SGSH), REP1, CYBB, RAG1, ADA, WAS, AC6, Factor VIII, HGF (hepatocyte growth factor) HGF728 and/or HGF723, SMN, CTRR, the Pfr1 gene for Perforin, Regulated expression of CAR-T and TCR receptors, SH2D1A for X-linked lymphoproliferative disease, BTK for X-linked agammaglobulinemia (XLA), ILR2-G for X-SCID, CD40L for X-Linked Hyper-IgM Syndrome, Il7Rg for SCID, CD3 delta for SCID, CD3 epsilon for SCID, CD3 zeta for SCID, ZAP70 for SCID, FOXP3 for IPEX, and the like.

These target genes are illustrative and non-limiting. Numerous other targets for gene therapy are well known to those of skill in the art and appropriate regulatory elements for these genes can readily be identified using the methods described herein.

It is also noted that the target gene need not be a target for gene therapy. Other target genes can be utilized, for example, for research purposes for agricultural and/or animal husbandry purposes.

(ii) Identifying One or a Plurality of Putative Regulatory Elements Associated with Said Target Gene;

Once a target gene or genes have been selected, one or a plurality of putative regulatory elements (e.g., enhancers) of that gene are identified. This is readily accomplished by the use of genomic databases that identify putative regulatory elements for such genes. Illustrative, but non-limiting examples of such databases include, but are not limited to 1) The ENCODE encyclopedia of DNA elements database (www.encodeproject.org/); 2) the ENSEMBL regulatory build database (e.g., version 92) which is available in the Ensembl genome browser (/uswest.ensembl.org/index.html); the FANTOM5 atlas of active enhancers (see, e.g., Andersson et al. (2014) Nature, 507(7493): 455-461; //fantom.gsc.riken.jp/5/); 4) The VISTA Enhancer Browser (e.g., Visel et al. (2007) 35(Database issue): D88-D92; //enhancer.lbl.gov/); 5) The dbSUPER database of super-enhancers (see, e.g., Kahn & Zhang (2016) Nucleic Acids Res., 44(D1):D164-171; //asntech.org/dbsuper/adv_search.php); 6) The Eukaryotic promoter database EPD (see, e.g., Dreos et al. (2015, Nucleic Acids Res., 43(Database issue): D92-96; //epd.epfl.ch/EPDnew_database.php); 7) The UCNEbase ultra-conserved noncoding elements; and the like.

It is known to those of skill in the art that a number of databases exist that aggregate data from one or more of these databases and/or other genomic databases. One illustrative, but non-limiting example is the GeneHancer database (see, e.g., Fishilevich et al. (2017) Database (Oxford) doi: 10.1093/database/bax028)). GeneHancer is a database of genome-wide enhancer-to-gene and promoter-to-gene associations, embedded in GeneCards as described herein in Example 1. The GeneHancer table lists a set of enhancers and promoters associated with the gene. Gene-GeneHancer associations and likelihood-based scores were generated using information that helps link regulatory elements to genes.

Accordingly in certain embodiments, one or a plurality of putative regulatory elements associated with the target gene are identified by querying one or more genomic databases (e.g., the databases identified above) that identify putative regulatory elements associated with genes identified in the one or more databases to identify said one or more putative regulatory elements.

It will be recognized that while typically the methods involve querying the database to identify one or one or a plurality of putative regulatory elements associated with the target gene comprises identifying one or more putative enhancers, in certain embodiments, the method can additionally or alternatively comprise identify a putative promoter and, as with enhancers, the key components of the promoter can be identified to produces reduced/slimmed promoter.

(iii) Determining if the Putative Regulatory Element is a Putative Key Regulatory Element,

As explained above, in various embodiments, for each of the identified putative regulatory elements, it is determined whether those regulatory elements are “key” regulatory elements. In certain embodiments this can be accomplished by determining the genomic coordinates of the putative regulatory element to identify putative boundaries of the regulatory element; and within those coordinates identifying a plurality of data tracks believed to be indicative of the regulatory component of the regulatory element.

In certain embodiments the data tracks include data tracks associated with gene expression and/or regulation. In certain embodiments the data tracks comprise data tracks available on project ENCODE and on the UCSC Genome Browser interface (see, e.g., list of data tacks at //genome.ucsc.edu/cgi-bin/hg/tracks which is incorporated herein by reference for the data tracks listed therein.). In certain embodiments the data tracks include, but need not be limited to epigenetic histone modifications, chromatin looping interactions, DNAse I hypersensitivity site, transcription factor binding site, and conservation of sequence across a plurality of vertebrates, and the like. In certain embodiments the data track comprises a data track selected from the group consisting of DNAse I hypersensitivity site, transcription factor binding site, and conservation of sequence across a plurality of vertebrates (e.g., 100 vertebrates sequence conservation).

While this is typically performed for each of the putative regulatory elements identified, it will be recognized that in certain embodiments this operation may be performed for one putative regulatory element, or a subset of putative regulatory elements.

The putative boundaries of the regulatory element in question can readily be identified by retrieving the genomic coordinates for the regulatory element from one or more of the databases identified above (e.g., using GeneCards comprising GeneHancer data).

In certain embodiments the coordinates of the regulatory element in question can be input into a genome browser (e.g., the UCSF genome browser) to identify the location(s) of data track(s) within that regulatory element.

Where the plurality of data tracks overlap in the genomic sequence the regulatory element is identified as a “key” regulatory element. Where there is no overlap of the data tracks of interest, the regulatory element is discarded and not considered a key regulatory element.

In certain embodiments the data track comprises a data track selected from the group consisting of DNAse I hypersensitivity site, transcription factor binding site, and conservation of sequence across a plurality of vertebrates (e.g., 100 vertebrates sequence conservation). In certain embodiments overlap of two or more data tracks is indicative that the regulatory element is a “key” regulatory element. In certain embodiments overlap of at least three or more data tracks is indicative that the regulatory element is a “key” regulatory element.

### (iv) Providing a List of the Key Regulatory Elements

The regulatory element identified as “key” regulatory elements are then “output to a user” (e.g., provided as a list of key regulatory elements). In certain embodiments the providing a list comprises providing the genomic coordinates of each of the key regulatory elements comprising the list. In certain embodiments the providing a list comprises providing output in a form selected from the group consisting of hard copy, data in a non-transitory computer readable memory, presentation of data on a display, provision of a datafile readable by a computer, provision of a data file readable by a DNA synthesizer. And output readable by a DNA vector design program. In certain embodiments the output comprises a file containing the sequence of the regulatory element. Such files can readily be read on Word, and molecular biology software such as Benchling, SnapGene, Gene Construction Kit, etc.

In certain embodiments the providing a list comprises providing output readable by a DNA vector design program to facilitate the design and construction of a vector comprising the identified key regulatory elements. Such vector design programs are well known to those of skill and include, but are not limited to GENESCRIPT®, VECTOR NTI®, GENSMART® DESIGN, SNAPGENE®, BENCHLING®, and the like.

### Construction of an Expression Cassette and a Vector Incorporating the Key Regulatory Elements.

In certain embodiments once the key regulatory elements are identified expression cassettes comprising one or more of those regulatory elements can be constructed to facilitate validation of the construct. Typically the expression cassette includes either the endogenous promoter or a modified promoter to drive expression of a gene or cDNA and one or more of the “key regulatory element” (e.g., enhancers). In certain embodiments the expression cassette includes the endogenous promoter for the gene of interest. In certain embodiments the expression cassette includes a slimmed promoter that comprises the key promoter elements for that promoter.

Typically the key regulatory elements (e.g., enhancers) are inserted in the expression cassette upstream of the promoter.

In various embodiments the expression cassette comprises the target gene or target gene cDNA or modified target gene to create a construct for expression of that gene.

In various embodiments the expression cassette comprise a reporter gene to facilitate validation of the construct.

In various embodiments the expression cassette is provided as a component of a gene therapy vector. Illustrative gene therapy vectors include, but are not limited to a lentiviral vector (LV), an adenovirus vector (AV), and an adeno-associated viral vector (AAV). In certain embodiments the gene therapy vector is a lentiviral vector (e.g., an HIV-1 vector).

### Validation of the Expression Cassette.

It is desirable to validate the vector (e.g., LV) comprising the expression cassette to determine which “key” regulatory elements are necessary to recapitulate the pattern of expression of the endogenous “target” gene. As shown in example 1, this can readily be accomplished by creating a library of vectors where each vector comprises a key regulatory element (e.g., enhancer) of interest and, in certain embodiments, a reporter gene (e.g., mcitrine, GFP, etc.) and screening the vectors in cell lines, and/or primary cells, or by transducing hematopoietic stem cells (HSC) and transplanting them into a mammalian test animal (e.g., NSG (NOD.Cg-Prkdcscid Il2rgtm1Wjl/SzJ) or BLT mice. In the case of transplanting the transduced HSCs into a subject (e.g., NSG/BLT mice), target cells, and/or target organs of interest (e.g., bone marrow, thymus, spleen, liver, peripheral blood, etc.) can be harvested post-transplant (e.g., at 16-20 weeks in mice) and different stage specific lineages can be FACS sorted.

In certain embodiments, as shown herein in Example 1, the transduced cells can include, but are not limited to neutrophils (e.g., mature neutrophils), monocytes, B cells, T cells and the like.

The transduced cells and tissues are then screened to determine expression level of the vectors any of a number of convenient means. Key regulatory elements that appear to be responsive of increased expression in the target cell(s) and/or tissues in which the endogenous gene is typically expressed can then readily be incorporated in an appropriate vector.

As indicated above, where multiple putative enhancers are identified an enhancer library can be produced by generating vectors where each vector contains an individual key regulatory element. In certain embodiments the vectors encode reporter genes. However, in certain embodiments, a known nucleotide sequence is added to the 3′ untranslated region of the vector, thereby associating each putative enhancer element to a specific barcode. Methods of generating and reading genetic barcodes are well known to those of skill in the art (see, e.g., Lyons et al. (2017) Scientific Reports 7: Article number: 13899; Morgan et al. (2020) Mol. Therap: Methods & Clinical Dev., 17: 999-1013; Shen et al. (2016) Genome Res., 26(2): 238-255; Patwardhan et al. (2012) Nat. Biotechnol. 30(3): 265-270; etc.).

This library (e.g., a lentiviral library) can then be screened, for example, in cell lines, in primary cells, or by transducing human CD34+ hematopoietic stem cells (HSCs) and transplanting them into a mammalian test animal (e.g., NSG (NOD.Cg-Prkdcscid Il2rgtm1Wjl/SzJ) or BLT mice. Cells or tissues of the test animal can be harvested at one or more time points post-transplant and different stage specific lineages can be FACS sorted. Genomic DNA (gDNA) and RNA can then be extracted from each population and the RNA cDNA converted which facilitates the sue of PCR (or other DNA amplification systems) to amplify out the barcodes from the gDNA and the RNA. By quantifying the abundance of barcodes in the RNA relative to the gDNA, the activity of each putative enhancer element in each cell population can be quantified.

In another illustrative, but non-limiting embodiment, the barcodes associated with each enhancer can be identified by skipping the FACs sorting and PCR steps and directly performing single cell RNA sequencing of the cells from each target organ. By RNA sequencing, it is possible to identify the cellular identity of each as well as quantify the abundance of barcodes in the transcriptome.

A lower throughput method of screening is to use flow cytometry to evaluate the expression of reporter gene in each construct across cell lines and/or primary cells. (this does not require barcodes).

After putative enhancer elements have been screened, the sequences of the validated enhancers of interest can then inputted back into a genome browser (e.g., the UCSC genome browser) and minimal boundaries are then defined (usually by DNaseI Hypersensitivity footprint). These “slimmed” enhancer elements that have been reduced in size are then compared to their original larger fragments to evaluate if enhancer activity has been retained. The enhancer element(s) (and in certain embodiments the promoter) are reduced in size with the goal of increasing titering while retaining expression. The validated and slimmed enhancer elements can then be combined together upstream of the minimal promoter of interest to generate a lead candidate vector for the disease of interest.

### EXAMPLES

The following examples are offered to illustrate, but not to limit the claimed invention.

### Example 1

### Design and Construction of a Highly Regulated Lentiviral Vector for the CYBB Gene

The field of viral based gene therapy currently uses 3rd generation lentiviral vectors, where expression of the transgene is now driven by any desired internal promoter or regulatory element. When translating this design to the clinic, the goal of this in the beginning was to obtain therapeutic levels of expression from this internal promoter without having the genotoxicity associated with the previously used gamma-retro viral vectors.

This led to the development and use of many constitutively active promoter elements such as EFS1u (the promoter of the human elongation factor 1 alpha), the viral derived MNDU3 (see, e.g., Logan et al. (2004) Hum Gene Ther. 15(10): 976-988), the promoter of the human Phosphoglycerate kinase (PGK) housekeeping gene, the promoter of the human Ubiquitin C (UBC) gene, as well as the use of ubiquitious chromatin opening element (UCOE) promoters (Zhang et al. (2010). Molecular Therapy, 18(9): 1640-1649).

Although, these approaches have displayed some success preclinically as well as clinically, a major limitation of using these constitutively active promoter elements is the lack of regulation and control of the gene being expressed (especially compared to the native gene).

We therefore employed a bioinformatics-guided approach to elucidate the native genomic elements which regulate any target gene of interest to design lentiviral vectors expressing transgenes driven by the endogenous elements which regulate it natively.

Here we describe the generation of an expression cassette for the CYBB gene and a gene therapy vector incorporating that expression cassette, where the cassette comprises the enhancer elements believed to be necessary to recapitulate endogenous expression of the CYBB gene.

Having identified CYBB as a target gene of interest, one or more putative regulatory elements associated with the gene were identified. This was accomplished by querying a genomic database that identifies putative regulatory elements. In this example, the GeneHancer database was queried although other databases could similarly be used. GeneHancer incorporates data from the ENCODE encyclopedia of DNA elements database, the Ensembl Regulatory Build database, the FANTOM5 atlas of active enhancers, the VISTA enhancer browser, the dbSUPER database of super enhancers, the eukaryotic promoter database (EPDnew), and the UCNEbase database.

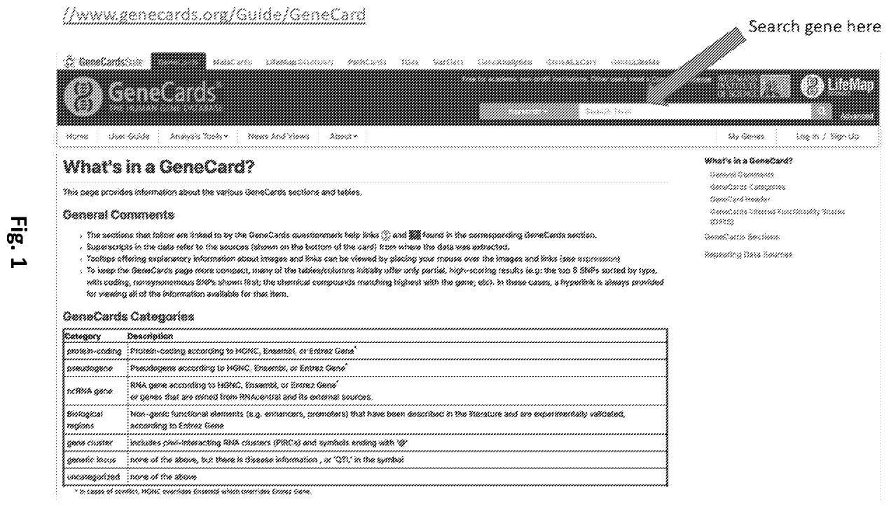

In the GeneHancer website embedded in GeneCards, it is possible to search essentially any target gene of interest (see, e.g., FIG. 1). Querying Genehancer for a particular gene brings up a table of the putative genomic regions regulating expression of that gene. In particular, the GeneHancer table lists a set of enhancers and promoters associated with the gene. Gene-GeneHancer associations and likelihood-based scores are generated using information that helps link regulatory elements to genes:
1) eQTLs (expression quantitative trait loci) from GTEx;
2) Capture Hi-C promoter-enhancer long range interactions;
3) Expression correlations between eRNAs and candidate target genes from FANTOM5;
4) Cross-tissue expression correlations between a transcription factor interacting with an enhancer and a candidate target gene; and
5) GeneHancer-gene distance-based associations, scored utilizing inferred distance distributions. Associations include several approaches: (a) Nearest neighbors, where each GeneHancer is associated with its two proximal genes (from all gene categories). In cases where a proximal gene is not protein coding, the nearest protein coding gene is also included; (b) Overlaps with the gene territory (Intragenic); (c) Proximity (<2 kb) to the gene TSS (transcription start site). TSS proximity scores are boosted to elevate Gene-GeneHancer associations in the vicinity of the gene TSS.

GeneHancer elements have unique, informative and persistent GeneHancer identifiers (GHids). The id begins with GH, which is followed by the chromosome number, a single letter related to the GeneHancer version (constant since version 4.8, ‘J’), and approximate kilobase start coordinate. For example, GHOXJ101383 is located on chromosome X, with starting position (in kb) of 101383.

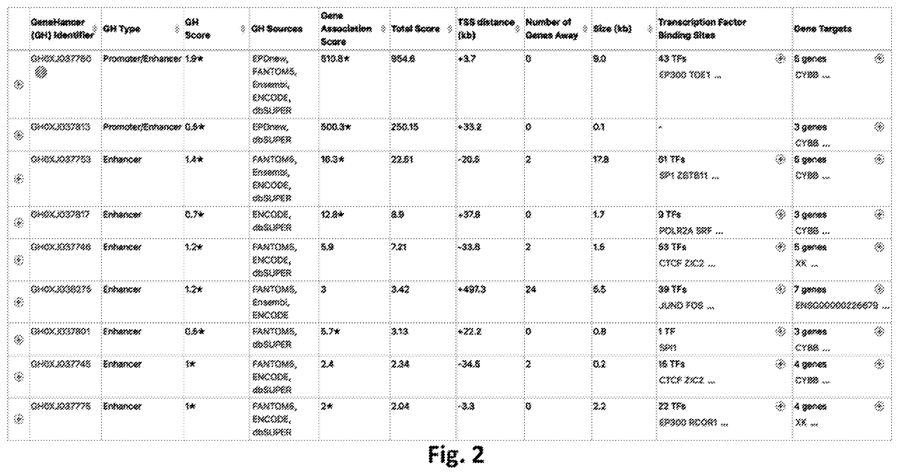

FIG. 2 provides a screenshot of the putative genomic regions regulating the CYBB gene obtained from the GeneHancer databases. There was a total of 25 regulatory regions identified.

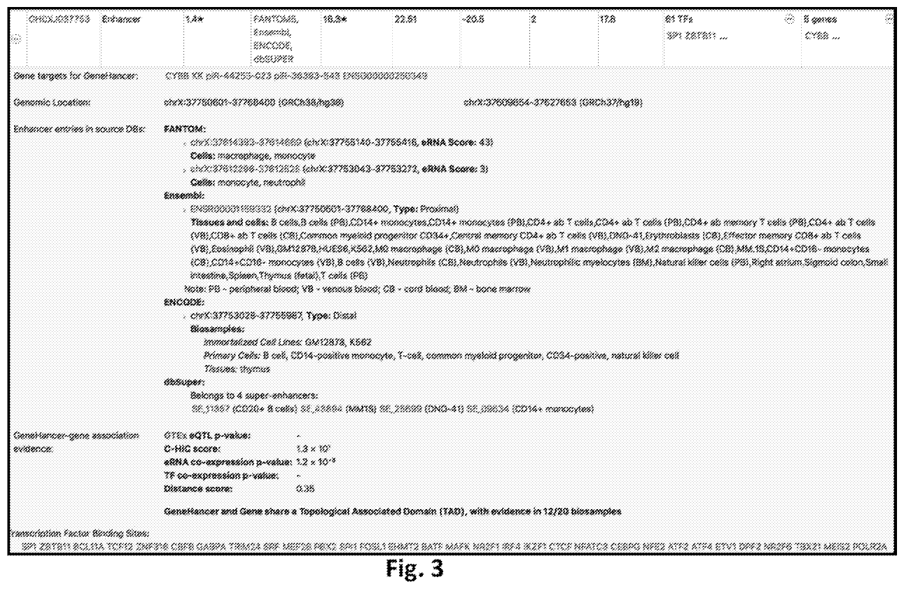

By clicking on each putative regulatory region in the GeneHancer database, GeneHancer provides more information and shows the genomic coordinates that the GeneHancer algorithm has flagged for each putative regulatory region (see, e.g., FIG. 3). This was done for all of the putative regulatory regions for a gene. In this case we did this for all 25 regions flagged by GeneHancer.

The coordinates for each putative regulatory region were then input into a genome browser. In the present example, the coordinates were input into the UCSC genome browser visual interface (//genome.ucsf.wedu/index.html) although other genome browsers could readily be utilized.

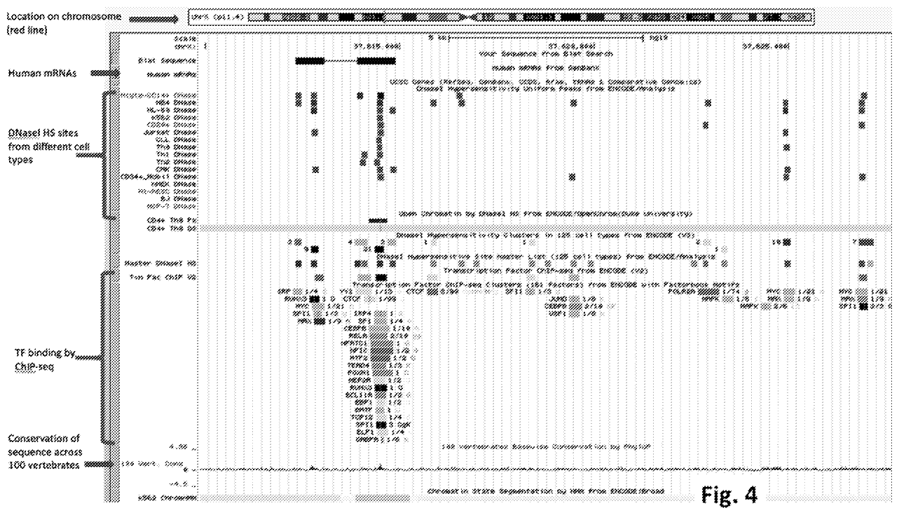

By inputting the coordinates of one specific regulatory region flagged by GeneHancer as an example, we were given ˜15 kb window in the human genome (see, e.g., FIG. 4). This region of the genome was too large to incorporate into a lentiviral vector so we needed need to find the key regulatory elements within this window to incorporate into the lentiviral vector.

Using specific data tracks such as DNaseI Hypersensitivity, Transcription Factor ChIP-seq and 100 vertebrates sequence conservation, we identified key regulatory elements within this 15 kb window. In particular, key regulatory elements were identified by overlap of specific data tracks as shown in FIG. 4.

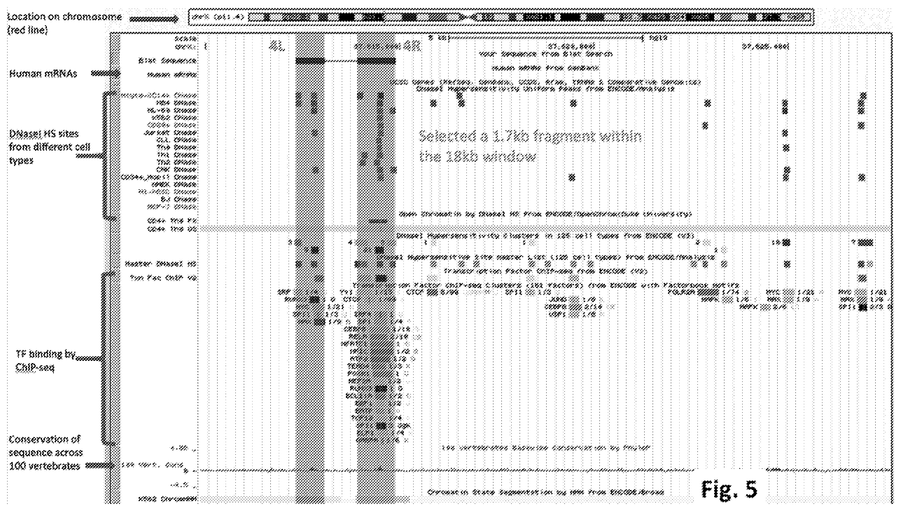

Using this approach the two key regulatory elements within this window (highlighted in red in FIG. 5) were identified. Essentially, a 1744 bp fragment was pulled out of the >15,000 bp window.

This was repeated for all the putative regulatory regions identified by GeneHancer (in this example there are 25 so we repeat this process 25 times). Note that some putative regulatory regions identified by GeneHancer do not contain any key regulatory elements using our analysis method. If so, we do not take anything from that genomic region. Through this analysis, 15 putative key regulatory elements were identified.

The putative enhancer elements were then experimentally validated. In order to experimentally identify the critical enhancer elements that regulate the CYBB gene, each putative enhancer element was cloned upstream of the endogenous CYBB promoter to drive expression of the mCitrine reporter gene (see, e.g., FIG. 6).

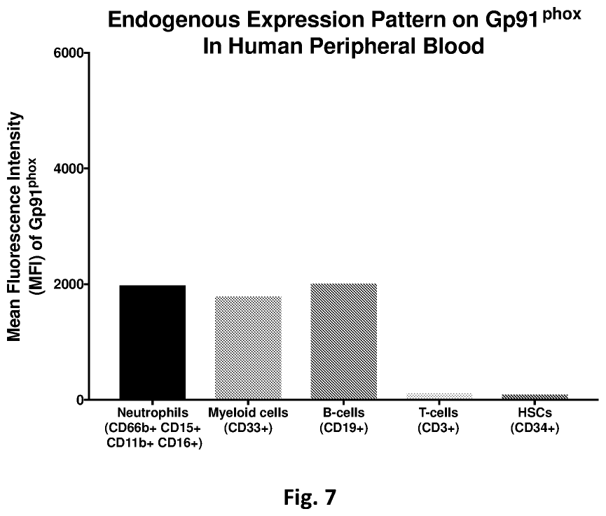

Each of the vectors was screened in multiple cell lineages (in primary cells as well as cell lines) to identify which enhancers are necessary for high-level expression in each cell lineage. The goal was to identify the enhancer necessary for high-level expression in each of the target cell lineages. In order to determine the target cell lineages of the endogenous gene target, we took human whole blood and used an antibody to stain for our protein of interest across different cellular lineages to identify the endogenous expression pattern for the CYBB gene. FIG. 7 shows the example of flow staining for Gp91^{phox}—the protein produced by the CYBB gene.

In particular, human whole blood was stained with an antibody against Gp91^{phox} to determine which cellular lineages the gene is naturally present in. The results shown in FIG. 7 indicate that high and equal levels of Gp91^{phox} are detected in the mature neutrophils, bulk myeloid cells and B-cell lineage with minimal expression in the T-cell lineage as well as in hematopoietic stem and progenitor cells (HSPCs).

From this, we now know which lineages to screen the putative enhancer elements in. Our hypothesis was that specific enhancers are necessary to high-level lineage specific expression. For example, we hypothesized that there is an enhancer necessary for driving high level of expression in neutrophils and another enhancer responsible for driving high-level expression in B-cells.

Since each enhancer element is driving the expression of an mCitrine reporter gene. We can measure how much mCitrine is expressed by each enhancer in each cell lineage to determine which enhancers are active in each cellular lineage.

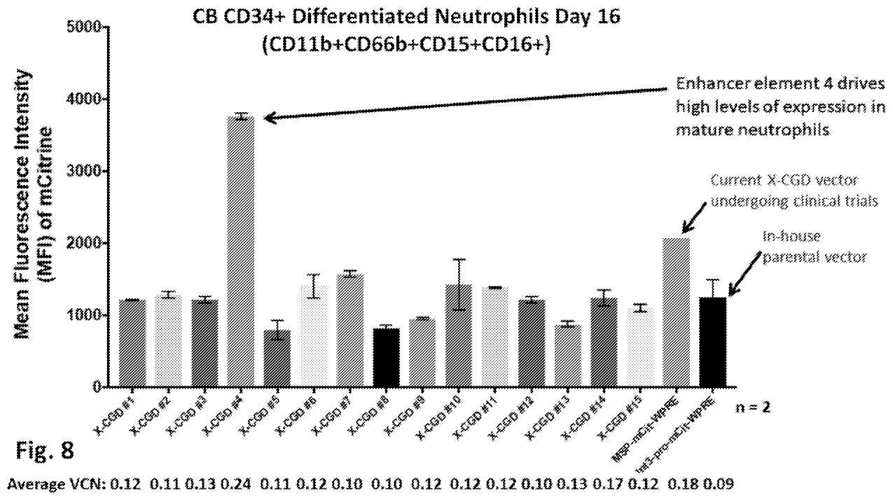

The results of the screen are shown in FIGS. 8-11. As shown in FIG. 8 in a mature neutrophil population, one element really pops up which is enhancer element number 4, expressing almost 2-fold higher than the current lentiviral vector undergoing clinical trials. 15 single element vectors listed, MSP is the currently clinical vector, Int3-pro-mCit-WPRE is the parental backbone to which were attached each of putative enhancer elements.

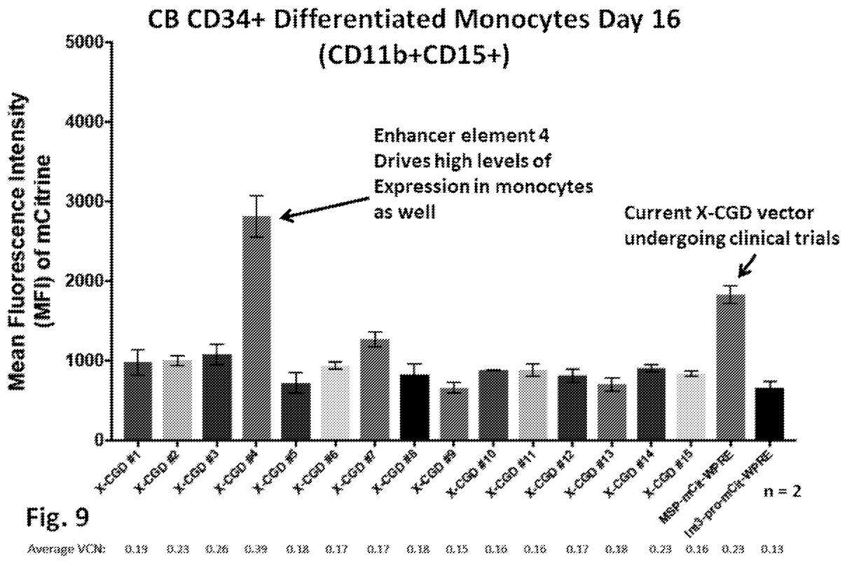

FIG. 9 shows that the same putative enhancer element 4 drives high levels of expression in monocytes. Again, this is higher than MSP and shows almost a 3-fold increase higher than the parental backbone suggesting that putative enhancer element 4 may be the myeloid specific element regulating the CYBB gene.

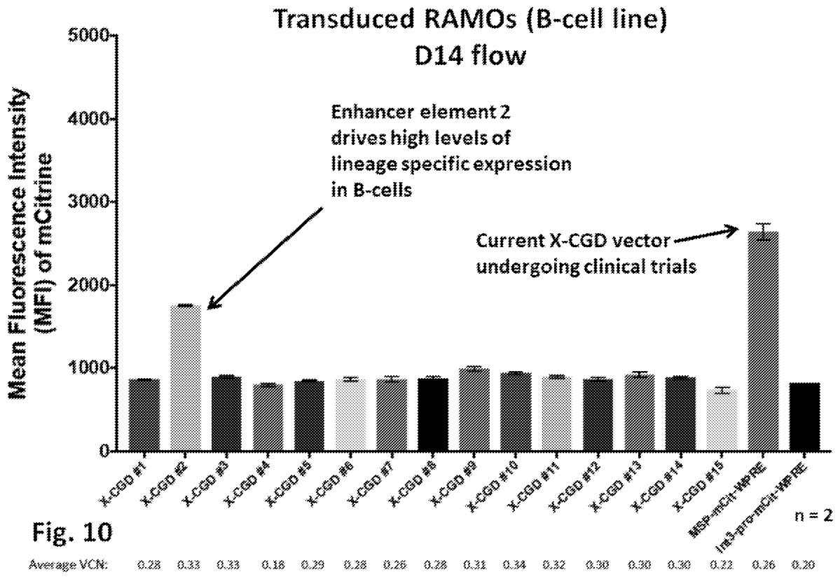

As shown in FIG. 10, in B-cells, element 4 is off, now element 2 is on driving expression. While expression is lower than MSP, it is about 2-fold higher than the parental backbone. Element 2 was off in the myeloid lineages but on in B-cells, while element 4 was on in the myeloid lineages but off in the B-cells suggesting these could be the endogenous elements necessary for high level myeloid and B-cell expression.

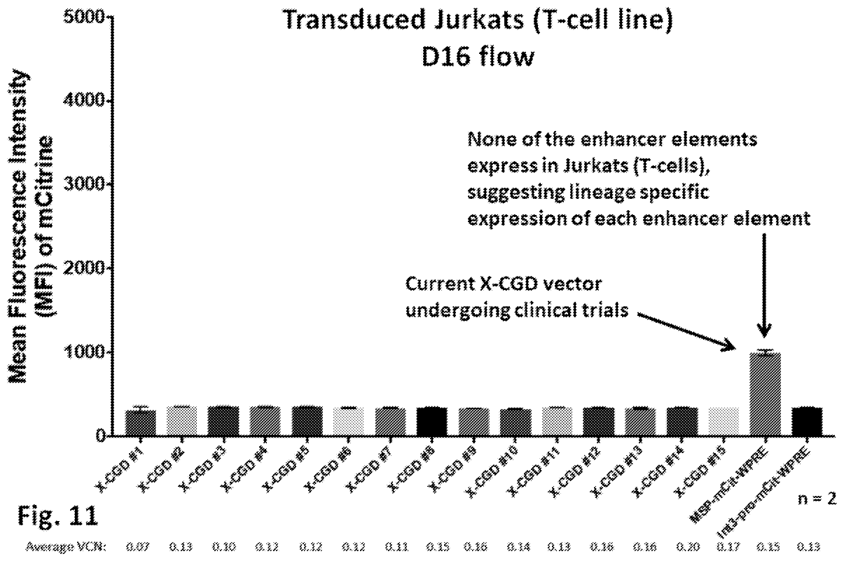

The lineage-specific expression of CYBB is clearly shown in the results of the T-cell screen (FIG. 11) where none of the elements are on and the most off-target expression is from the current clinical vector.

From this preliminary enhancer screen we determined the following:
1) Element 4 is an endogenous enhancer element necessary for driving high-level lineage specific expression in mature neutrophils and monocytes;
2) Element 2 is an endogenous enhancer element necessary for driving high-level lineage specific expression in B-cells; and
3) Both of these elements have no expression in the off-target T-cell lineage

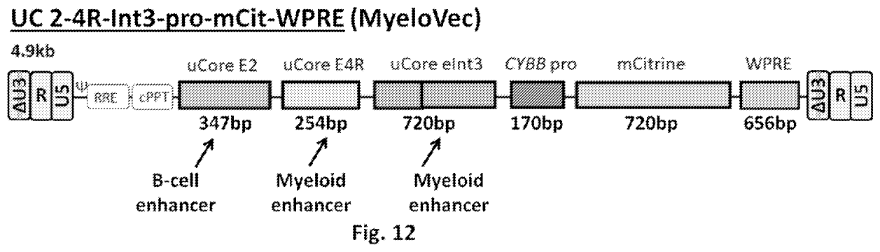

Using the results of this screen, we combined enhancer elements 4 and 2 to generate a vector possessing strict lineage specific expression in the neutrophil and B-cell lineages, recapitulating the endogenous expression pattern of the CYBB gene shown in FIG. 7. The resulting vector is illustrated in FIG. 12.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of designing an expression cassette that recapitulates expression patterns for a target gene when introduced into a mammal, said method comprising:
(i) selecting a target gene;
(ii) identifying one or a plurality of putative regulatory elements associated with said target gene;
(iii) for each putative regulatory element identified in step (ii) determining if said regulatory element is a key regulatory element by:
a) determining the genomic coordinates of said putative regulatory element to identify putative boundaries of said putative regulatory element;
b) within said coordinates identifying a plurality of data tracks; and
c) when the genomic coordinates of at least two of said data tracks overlap, identifying said regulatory element as a key regulatory element; and
(iv) providing a list of the key regulatory elements identified in step (iii).

2. The method of claim 1, wherein said selecting a target gene comprises selecting a gene associated with a genetic disorder.

3. The method according to any one of claims 1-2, wherein said target gene comprises a gene selected from the group consisting of β-globin gene, Factor IX (FIX), human fibroblast growth factor-4 (FGF-4), ND4, ABCD1, N-sulfoglucosamine sulfohydrolase (SGSH), REP1, CYBB, RAG1, ADA, WAS, AC6, Factor VIII, HGF (hepatocyte growth factor) HGF728 and/or HGF723, SMN, and CTRR.

4. The method according to any one of claims 1-3, wherein said identifying one or a plurality of putative regulatory elements associated with said target gene comprises querying one or more genomic databases that identify putative regulatory elements associated with genes identified in said one or more databases to identify said one or more putative regulatory elements.

5. The method of claim 4, wherein said identifying one or a plurality of putative regulatory elements associated with said target gene comprises querying one or more genomic databases selected from the group consisting of the ENCODE encyclopedia of DNA elements database, the Ensembl Regulatory Build database, the FANTOM5 atlas of active enhancers, the VISTA enhancer browser, the dbSUPER database of super enhancers, the eukaryotic promoter database (EPDnew), and the UCNEbase database, and/or database incorporating date from one or more of these databases.

6. The method of claim 5, wherein said identifying one or a plurality of putative regulatory elements associated with said target gene comprises querying a database incorporating data from a plurality of the databases.

7. The method of claim 6, comprises querying the GeneHancer database.

8. The method according to any one of claims 4-7, wherein said wherein said identifying one or a plurality of putative regulatory elements associated with said target gene comprises identifying a promoter.

9. The method according to any one of claims 4-8, wherein said wherein said identifying one or a plurality of putative regulatory elements associated with said target gene comprises identifying one or more enhancer(s).

10. The method according to any one of claims 1-9, wherein said determining the genomic coordinates of said regulatory element to identify putative boundaries of said regulatory element comprises retrieving said genomic coordinates from one or more genomic databases.

11. The method according to any one of claims 1-10, wherein said identifying a plurality of data tracks comprises inputting genomic coordinates of said putative enhancer into a genome browser and identifying overlapping data tracks in said genome browser.

12. The method of claim 11, wherein said genome browser comprises the UCSC genome browser.

13. The method according to any one of claims 1-12, wherein said identifying a plurality of data tracks comprises identifying a plurality of data tracks associated with gene expression and/or gene regulation.

14. The method according to any one of claims 1-13, wherein said plurality of data tracks comprise one or more data tracks selected from the group consisting of epigenetic histone modifications, chromatin looping interactions, DNAse I hypersensitivity site, transcription factor binding site, and conservation of sequence across a plurality of vertebrates.

15. The method according to any one of claims 1-13, wherein said plurality of data tracks comprise one or more data tracks selected from the group consisting of DNAse I hypersensitivity site, transcription factor binding site, and conservation of sequence across a plurality of vertebrates.

16. The method according to any one of claims 1-15, wherein said identifying a plurality of data tracks comprises identifying a DNAse I hypersensitivity site within said coordinates.

17. The method according to any one of claims 1-16, wherein said identifying a plurality of data tracks comprises identifying a transcription factor binding site within said coordinates.

18. The method according to any one of claims 1-17, wherein said identifying a plurality of data tracks comprises identifying a sequence conservation across a plurality of vertebrates.

19. The method according to any one of claims 1-18, wherein upon determining overlap of the data tracks DNAse I hypersensitivity site, transcription factor binding site, and conservation of sequence across a plurality of vertebrates, labeling regulatory element as a key regulatory element.

20. The method according to any one of claims 1-19, wherein said conservation of sequence across a plurality of vertebrates comprises 100 vertebrates sequence conservation.

21. The method according to any one of claims 1-20, where said providing a list comprises providing the genomic coordinates of each of the key regulatory elements comprising said list.

22. The method of claim 21, wherein said providing a list comprises providing output in a form selected from the group consisting of hard copy, data in a non-transitory computer readable memory, presentation of data on a display, provision of a datafile readable by a computer, provision of a data file readable by a DNA synthesizer. And output readable by a DNA vector design program.

23. The method of claim 22, wherein said providing a list comprises providing output readable by a DNA vector design program.

24. The method of claim 22, wherein said providing a list comprises providing output readable by a DNA vector design program selected from the group consisting of GENESCRIPT®, VECTOR NTI®, GENSMART® DESIGN, BENCHLILNG, and SNAPGENE®.

25. The method according to any one of claims 1-24, wherein said method comprises assembling an expression cassette where said expression cassette comprises:
a gene or cDNA to be expressed by said cassette;
a promoter comprising an endogenous promoter for said gene, or a reduced promoter comprising key regulatory elements comprising said promoter; and
one or more key regulatory elements that are not elements of said promoter selected from said list of key regulatory elements.

26. The method of claim 25, wherein said expression cassette comprise an endogenous promoter for said gene.

27. The method of claim 25, wherein said expression cassette comprises a reduced promoter comprising key regulatory elements comprising said promoter.

28. The method according to any one of claims 25-27, wherein said expression cassette comprises a key regulatory element comprising an enhancer where said enhancer is disposed upstream from said promoter.

29. The method according to any one of claims 25-28, wherein said gene or cDNA comprises a reporter gene.

30. The method of claim 29, wherein said reporter comprises a reporter gene selected from the group consisting of mcitrine, mStrawberry, green fluorescent protein (GFP), yellow fluorescent protein (YFP), and red fluorescent protein (RFP).

31. The method according to any one of claims 25-28, wherein said gene or cDNA comprises said target gene or a cDNA of said target gene, or a modified form of said target gene or target gene cDNA.

32. The method of claim 31, wherein said gene or cDNA comprises a gene selected from the group consisting of β-globin gene, anti-sickling β-globin (βAS3-FB), Factor IX (FIX), human fibroblast growth factor-4 (FGF-4), ND4, ABCD1, N-sulfoglucosamine sulfohydrolase (SGSH), REP1, CYBB, RAG1, ADA, WAS, AC6, Factor VIII, HGF (hepatocyte growth factor) HGF728 and/or HGF723, SMN, CTRR, BTK, ILR2-G, CD40L, Il7Rg, CD3 delta, CD3 epsilon, CD3 zeta, ZAP70, and FOXP3.

33. The method according to any one of claims 25-32, wherein said method comprises providing said expression cassette in a gene therapy vector.

34. The method of claim 33, wherein method comprises providing said expression cassette in a gene therapy vector selected from the group consisting of a lentiviral vector (LV), an adenovirus vector (AV), and an adeno-associated viral vector (AAV).

35. The method of claim 33, wherein said gene therapy vector is a lentiviral vector.

36. The method of claim 35, wherein said vector is an HIV-1 lentiviral vector.

37. The method according to any one of claims 33-36, wherein said gene therapy vector includes a known nucleotide sequence in the 3′ untranslated region of said vector where a unique known DNA barcode sequence is provided for each putative enhance in said expression cassette.

38. The method according to any one of claims 33-37, wherein said vector is introduced into a mammalian cell and the expression level of the gene or cDNA encoded in the vector is determined.

39. The method of claim 38, wherein said vector is transduced into a primary cell line.

40. The method of claim 38, wherein said vector is transduced into a cell line.

41. The method of according to any one of claims 38-40, wherein said method comprises quantifying expression of said gene or cDNA in said primary cell or cell line where elevated level of expression of said gene indicates that said putative enhancer(s) are valid/effective enhancer(s).

42. The method of claim 41, wherein said method comprises quantifying expression of a reporter gene.

43. The method of claim 41, wherein said method comprises quantifying expression of a reporter gene using flow cytometry.

44. The method of claim 38, wherein said vector is transduced into a cell that is transplanted back into a test mammal.

45. The method of claim 44, wherein said vector is transduced into a hematopoietic stem cell (HSC).

46. The method of claim 45, wherein said vector is transduced into a CD34+ hematopoietic stem cell.

47. The method according to any one of claims 44-46, wherein said vector is transduced into a mammal selected from the group consisting of a mouse, a rat, a rabbit, a porcine, a canine, a camelid, and a non-human primate.

48. The method of claim 47, wherein said vector is transduced into a mouse.

49. The method of claim 48, wherein said vector is transduced into an NSG or a BLT mouse.

50. The method according to any one of claims 44-49, wherein cells or tissues or organs are harvested from said animal at one or more time points after transduction of said vector into said mammal quantification of vector nucleic acid in said cells, tissues, or organs to quantify the activity of the putative enhancer(s) in each cell population where an elevated expression level of said vector compared to the genomic DNA level indicates that said putative enhancer(s) are valid/effective enhancer(s).

51. The method of claim 50, wherein said method comprises extracting genomic DNA and RNA, converting the RNA to cDNA, and using DNA amplification to amplify barcodes from the gDNA and RNA and quantifying the abundance of bar codes in the RNA relative to the gDNA.

52. The method of claim 50, wherein said method comprises direct single cell RNA sequencing of cells from said mammal to identify cellular identity and to quantify the abundance of barcodes in the transcriptome of said cell.

53. The method according to any one of claims 41 and 50, wherein said method comprises viewing data tracks for the enhancers identified as valid/effective enhancers and defining minimal boundaries for each of the enhancer(s) to produce slimmed enhancers.

54. The method of claim 53, wherein said minimal boundaries are defined by a DNaseI hypersensitivity data tracks.

55. The method of claim 53, wherein said method comprises placing one or more of said slimmed enhancers upstream of said promoter or reduced promoter to generate a lead candidate vector.

56. A vector designed according to the method according to any one of claims 1-55.

57. The vector of claim 56, wherein said vector is a lentiviral vector.
